(19) United States
(12) Patent Application Publication
(10) Pub. No.: US 2024/0265512 A1
(43) Pub. Date: Aug. 8, 2024

(54) SYSTEMS AND METHODS FOR AUTOMATIC SUPPRESSION OF COMET TAIL ARTIFACTS IN B-MODE IMAGES OF AN INTRACARDIAC ABLATION CATHETER

(71) Applicant: yoR Labs, Inc., Beaverton, OR (US)

(72) Inventors: Larry Y.L. Mo, North Potomac, MD (US); Greg Bashford, Lincoln, NE (US); Joseph R. Cole, Beaverton, OR (US); Airidas Korolkovas, Houston, TX (US); Anshumali Roy, Portland, OR (US); Dan Oliver, Beaverton, OR (US); Lihong Pan, Brookfield, WI (US)

(21) Appl. No.: 18/430,139

(22) Filed: Feb. 1, 2024

Related U.S. Application Data
(60) Provisional application No. 63/482,953, filed on Feb. 2, 2023.

(51) Int. Cl. G06T 5/80; A61B 8/00; A61B 8/08; G06T 7/00

(52) U.S. Cl. CPC G06T 5/80; A61B 8/469; A61B 8/5269; G06T 7/0012; G06T 2207/10132; G06T 2207/30048

(57) ABSTRACT

Systems and methods are disclosed for reducing a comet tail aberration in an image of an ablation catheter. In one example, the method includes receiving ICE images depicting the ablation catheter tip and an area proximate to the ablation catheter tip; automatically determining, by the computer system, a location of the tip of the ablation catheter in the received ICE images; automatically determining, by the computer system, the presence of a comet tail aberration in a region of interest (ROI) near the ablation catheter tip in the received ICE images; and in response to determining the aberration is present in the ROI, automatically changing, by the computer system, an imaging characteristic of the ICE imaging system to affect the appearance of the aberration in the ROI in subsequent images generated by the ICE imaging system using the changed imaging characteristic.

### INCORPORATION BY REFERENCE TO ANY PRIORITY APPLICATIONS

This application claims the benefit of U.S. Provisional Application No. 63/482,953, filed Feb. 2, 2023, which is incorporated by reference herein in its entirety. Any and all applications for which a foreign or domestic priority claim is identified in the Application Data Sheet as filed with the present application are hereby incorporated by reference under 37 CFR 1.57 for all purposes and for all that they contain.

### BACKGROUND

Under certain environmental or use conditions, intracardiac echocardiography (ICE) images of intracardiac ablation catheters can include aberrations that obscure the tip of the catheter such that it is difficult to determine the exact location of the tip. In ablation procedures, understanding the location of the catheter tip is extremely important for using its ablation functionality effectively. Accordingly, reducing or eliminating aberrations in images of the catheter tip are advantageous for its safe and effective use.

### SUMMARY

This disclosure relates to systems, methods, and techniques for automatic suppression of an imaging artifact on a B-mode intracardiac echocardiography (ICE) image. Various embodiments of systems, methods and devices within the scope of the appended claims each have several aspects, no single one of which is solely responsible for the desirable attributes described herein. Without limiting the scope of the appended claims, some prominent features are described herein.

Intracardiac echocardiography (ICE) uses a transducer arranged on a catheter to generate images from within the heart. The transducer of the ICE catheter is configured to image at short distances with high spatial resolution. Sometimes an ICE catheter may be placed inside a large vessel within the heart, in which case it can be described as a form of “intra-vascular ultrasound imaging (IVUS).” However, an IVUS system usually refers to a different ultrasound imaging technology designed to image arterial plaques inside major arteries including the coronary arteries. Some interventional cardiology ultrasound systems are designed to support both ICE and IVUS catheters. Although this disclosure refers to ICE imaging systems and ICE images, the systems and methods in this disclosure can be applicable to both ICE imaging and IVUS imaging, that is, such systems and methods can be used regardless of the name, or intended use, of a particular ultrasound catheter.

During certain procedures, images produced by an ICE imaging system can include an aberration which obscures important information in the image. For example, during a radiofrequency (RF) ablation procedure using a RF ablation catheter, an ICE imaging system can be deployed near the RF ablation catheter to monitor the ablation process in real time, generating ICE images that can include the RF ablation catheter tip and a portion of the tissue being ablated. In some circumstances, due to physical characteristics of the ultrasound imaging process and surfaces being imaged, ICE images displayed to the medical practitioner performing the procedure may include a bright line artifact between the RF ablation catheter tip and tissue being ablated. While practitioners may refer to the artifact between the RF ablation catheter tip and tissue being ablated as a “comet tail,” some medical practitioners may just refer to this artifact as an aberration or an artifact. For ease of reference in this disclosure, the term “comet tail” or “comet tail aberration” or “comet tail artifact” is used to refer to such an aberration or artifact. However, as one of ordinary skill in the art will appreciate, the methods and systems disclosed herein apply to addressing this aberration whether it is referred to as a comet tail, an artifact, or an aberration, or another name. The comet tail aberration can be a result of, for example, multiple ultrasound reverberations within the catheter tip, such that the delayed echoes from the multiple reverberations appear as reflections along the ultrasound beam but extending beyond the catheter tip. Comet tail aberration often makes it difficult to follow the precise location of the all-important intracardiac ablation catheter tip, and thus can impact the efficiency and effectiveness of the procedure. Accordingly, it is advantageous to minimize the appearance for a comic tail aberration in the displayed images to allow for clearer information to be provided to the medical practitioner by the ICE images. It is also advantageous to minimize or prevent the formation of a comet tail aberration such that the ICE images of the ablation catheter tip are as clear as possible for the medical practitioner.

This disclosure describes methods for producing ICE images with reduced comet tail aberrations, and preferably no comet tail aberrations. This disclosure also describes methods of removing comet tail aberrations from ICE images. For example, in some embodiments, the control system which is processing data received from an intracardiac ICE imaging system, is configured to determine the presence of a comet tail aberration in an ICE image. In response to determining the presence of the comet tail aberration, the control system may change one or more imaging parameters of the ICE imaging system to minimize or remove comet tail aberrations from subsequent images. In some embodiments, in response to determining the presence of the comet tail aberration, the control system which is processing data received from an intracardiac ICE imaging system is configured to perform image processing on the received ICE images to remove or reduce the appearance of the comet tail aberration.

Accordingly, one innovation includes a computer system implemented method of reducing a comet tail aberration in an image of an ablation catheter. In some embodiments, the method includes receiving, by the computer system from an intracardiac echocardiography (ICE) imaging system on a catheter coupled to the computer system, a first set of images depicting the ablation catheter tip and an area proximate to the ablation catheter tip, the first set of images including at least one ICE image; automatically determining, by the computer system, a location of the tip of the ablation catheter in the first set of images; automatically determining, by the computer system, the presence of a comet tail aberration in a region of interest (ROI) near the ablation catheter tip in the first set of images; and in response to determining the aberration is present in the ROI, automatically changing, by the computer system, an imaging characteristic of the ICE imaging system to affect the appearance of the aberration in the ROI in subsequent images generated by the ICE imaging system using the changed imaging characteristic.

Such methods can include one or more different aspects. Changing the imaging characteristic can include changing a scan parameter used to generate the first ICE image set. Changing a scan parameter can include changing a transmission focus location of the ICE imaging system, changing a transmission frequency of the ICE imaging system, changing a fundamental transmission frequency of the ICE imaging system, changing a transmission frequency for the tissue harmonic mode of the ICE imaging system, ICE and/or changing a transmission pulse of the ICE imaging system. In some embodiments, changing the transmission pulse involves changing a single pulse sequence transmitted by the ICE imaging system. In some embodiments, the changing the transmission pulse comprises changing excitation pulses transmitted by the ICE imaging system. In some embodiments, changing the imaging characteristic comprises changing a beamforming method of the ICE imaging system. In some embodiments, changing the imaging characteristic comprises changing a beamforming method tuned towards reverberation noise isolation. In some embodiments, changing the imaging characteristic comprises changing a beamforming method to a model-based image reconstruction approach tuned towards wavefront or reverberation isolation and suppression. In some embodiments, the method includes receiving ICE images from the ICE imaging system that were generated using the changed imaging characteristic; automatically determining in the ROI, in images generated by the ICE imaging system using the changed imaging characteristic, if the aberration has been reduced as a result of the changed imaging characteristic, and in response to determining that the aberration has not been reduced, automatically changing a second imaging characteristic of the ICE imaging system to change the appearance of the comet tail aberration in the ROI in subsequent images generated by the ICE system using second imaging characteristic. In some embodiments, the method further includes generating and displaying a representation of the ICE images.

In some embodiments of such methods, the method includes in response to a user input, changing a second imaging characteristic of the ICE imaging system to change the appearance of the comet tail aberration in the ROI in subsequent images generated by the ICE system using the second imaging characteristic. In some embodiments of such methods, wherein automatically determining, by the computer system, the presence of the comet tail aberration in the ROI comprises accessing predetermined information of the ablation catheter characteristics. In some embodiments, wherein automatically determining, by the computer system, the presence of the comet tail aberration in the ROI comprises using reverberation wavefront modeling. In some embodiments, automatically determining, by the computer system, the presence of the comet tail aberration in the ROI comprises comparing ICE images generated using two different frequencies, determining which frequency is associated with images that have less of an appearance of a comet tail aberration in the image, and selecting the frequency that is associated with images that have less of an appearance of a comet tail aberration in the image for use in subsequent ICE images. In some embodiments, automatically determining, by the computer system, the presence of the comet tail aberration in the ROI comprises comparing ICE images generated using two different imaging modes. In some embodiments, automatically determining, by the computer system, the presence of the comet tail aberration in the ROI comprises using one or more morphological or image segmentation filters for yes/no feature identification images that have less of an appearance of a comet tail aberration in the image. In some embodiments, automatically determining, by the computer system, the presence of the comet tail aberration in the ROI comprises using a machine learning (ML) process (e.g., basic convolution neural network (CNN), reverb physics-informed AI).

Another innovation includes a non-transient computer readable medium containing program instructions for causing a computer to perform the method of reducing a comet tail aberration in an image of an ablation catheter, the method comprising under control of a computing system comprising one or more computer processors configured to execute specific instructions, receiving at the computer system an intracardiac echocardiography (ICE) imaging system on a catheter coupled to the computer system, a first set of images depicting the ablation catheter tip and an area proximate to the ablation catheter tip, the first set of images including at least one ICE image; automatically determining, by the computer system, a location of the tip of the ablation catheter in the first set of images; automatically determining, by the computer system, the presence of a comet tail aberration in a region of interest (ROI) near the ablation catheter tip in the first set of images; and in response to determining the aberration is present in the ROI, automatically changing, by the computer system, an imaging characteristic of the ICE imaging system to affect the appearance of the aberration in the ROI in subsequent images generated by the ICE imaging system using the changed imaging characteristic.

Another innovation includes a computer system implemented method of reducing a comet tail aberration in an image of an ablation catheter. The method includes receiving and displaying a first ICE image, wherein the first ICE image is generated with first imaging characteristics; determining the location of a catheter tip in the first ICE image; determining the presence of a comet tail aberration, associated with the catheter tip, in a region of interest (ROI) in the ICE image; in response to determining the presence of the comet tail aberration, initiating a dual frequency B-mode scanning sequence where a first frequency is used to generate a second ICE image in a first region of first region of interest (ROI) that covers the catheter tip and a second region of interest (ROI) that includes a tissue ablation region; processing the second ICE image to generate a smoothed ICE image, the smoothed ICE image having smoothed boundaries between a background region, the first ROI, and the second ROI; and displaying the smoothed second ICE image.

Another innovation includes a computer system implemented method of reducing a comet tail aberration in an image of an ablation catheter, the method comprising receiving and displaying a first ICE image, wherein the first ICE image is generated with first imaging characteristics, determining the location of a catheter tip in the first ICE image, determining the presence of a comet tail aberration, associated with the catheter tip, in a region of interest (ROI) in the ICE image, in response to determining the presence of the comet tail aberration, initiating a dual frequency B-mode scanning sequence where a first frequency is used to generate a normal background ICE image with the desired image quality in terms of spatial resolution and penetration, and a second frequency is used for a special region of interest (ROI) that covers the catheter tip and a tissue ablation region; the second frequency is chosen specifically to minimize the comet tail artifact even if the general image quality may be compromised (within an acceptable level); blend the image regions generated by the two frequencies together by smoothing the boundaries between the background region and the special ROI, and displaying the blended ICE image.

Additional embodiments of the disclosure are described below in reference to the appended claims, which may serve as an additional summary of the disclosure.

In various embodiments, systems and/or computer systems are disclosed that comprise a computer readable storage medium having program instructions embodied therewith, and one or more processors configured to execute the program instructions to cause the one or more processors to perform operations comprising one or more aspects of the above-and/or below-described embodiments (including one or more aspects of the appended claims).

In various embodiments, computer-implemented methods are disclosed in which, by one or more processors executing program instructions, one or more aspects of the above-and/or below-described embodiments (including one or more aspects of the appended claims) are implemented and/or performed.

In various embodiments, computer program products comprising a computer readable storage medium are disclosed, wherein the computer readable storage medium has program instructions embodied therewith, the program instructions executable by one or more processors to cause the one or more processors to perform operations comprising one or more aspects of the above-and/or below-described embodiments (including one or more aspects of the appended claims).

### BRIEF DESCRIPTION OF FIGURES

These and other features, aspects, and advantages of the present application are described with reference to drawings of certain embodiments, which are intended to illustrate, but not limit, the present disclosure. It is to be understood that the attached drawings are for the purpose of illustrating concepts disclosed in the present application and may not be to scale:

FIG. 1 is a block diagram of an example of an ICE imaging system.

FIG. 2 is a schematic that illustrates an example of an ICE imaging system that is used to image an RF ablation catheter tip and tissue being ablated.

FIG. 3 is an example of an ICE image produced by an ICE imaging system illustrating the RF ablation catheter and the comet tail aberration.

FIG. 4 is an example of an ICE image generated at a 6 MHz frequency setting well matched to the transducer center frequency and illustrating reverberation noise (a very short comet tail in this example) along the ultrasound scan line beyond the catheter tip.

FIG. 5 is an example of an ICE image generated at an 8 MHz frequency setting, which is close to the upper band edge of the ICE transducer response.

FIG. 6 is another example of an ICE image illustrating a comet tail between the location of the catheter tip and tissue near the catheter tip, where the ICE image is generated using multiple frequencies.

FIG. 7 is a flowchart of a process for minimizing or reducing a comet tail aberration in an ICE image.

FIG. 8 is a block diagram that illustrates a computer system that may be used in some embodiments of this disclosure.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims that may arise here from is not limited by any of the embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The following is a list of certain components that are described and enumerated in this disclosure in reference to the above-listed figures. However, any aspect of the devices illustrated in the figures, whether or not named out separately herein, can form a portion of various embodiments of the systems, devices, and methods described herein and may provide basis for claim limitation relating to such aspects, with or without additional description. The enumerated components include:
101 ICE imaging system
102 ICE image
104 ICE catheter
105 ICE imaging element (e.g., transducer)
106 ultrasound transmitted energy
108 ablation catheter
109 ablation tip/ablation element (e.g., RF, laser)
110 ablation energy
112 ablation 114 ICE handle/controller
116 control system
120 display
122 connection between handle and control system
123 connection between control panel and control system
124 connection between control system and display
130 comet tail
150 tissue (subject of ablation)
160 grating lobe noise
170 first zone (e.g., low frequency imaging region)
180 second zone (e.g., high frequency imaging region)
190 third zone (e.g., a second low frequency imaging region)

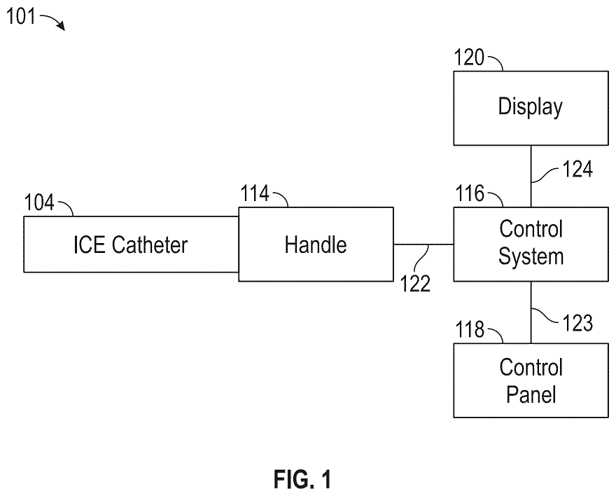

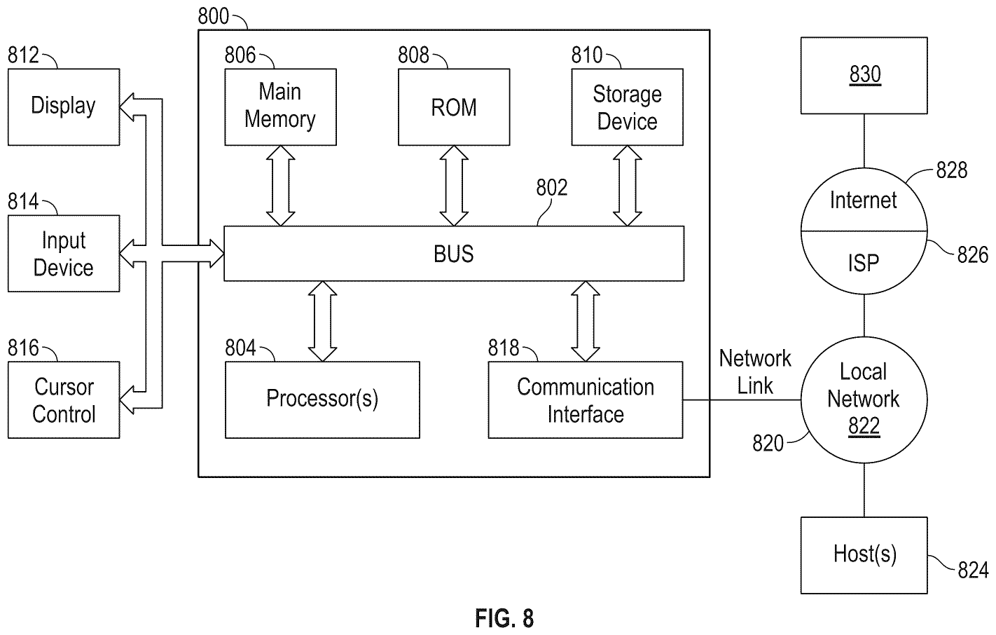

FIG. 1 is a block diagram of an example of an ICE imaging system 101. ICE imaging systems may include one or more additional components, or subcomponents, which are not illustrated in FIG. 1 for clarity of the illustration. In this example, the ICE imaging system 101 includes a control system 116, which is coupled to a handle (controller) 114 via a connection 122 configured to communicate signals from the control system 116 to the handle 114 and/or the ICE catheter 104, and communicate imaging information from the ICE catheter 104 to the control system 116. The connection 122 may also communicate information from the handle 114 to the control system 116 (e.g., status, operational information, etc.). The control system 116 is also coupled to a display 120 via a connection 124. The connection 124 between the display 120 and the control system 116 can be a wireless connection or a wired connection. the ICE imaging system 101 can also include a control panel 118 for controlling functionality associated with the control system 116, attached catheter, and/or display 120. In some embodiments, the control panel 118 can be incorporated into the control system 116. In some embodiments, the control panel 118 can be housed separate from the control system 116 and communicate with the control system 116 via a wired or wireless connection 123. The control panel 118 can include a display and a user interface. In some embodiments, the display is a touchscreen and at least a portion of the user interface is accessed via the touchscreen. The handle 114 is connected to an intracardiac ICE catheter 104. The ICE 104 includes an ICE imaging element (e.g., a transducer) which is used generated ICE images. The connection 122 is used to communicate signals to the ICE to the ICE catheter 104, via the handle 114, and to receive ICE image information from the ICE catheter 104 (via the handle 114) and is typically a wired connection. However, in some embodiments connection 122 can be a wireless connection. The handle 114 may include mechanical and/or electrical control components for operating the ICE catheter 104. For example, the handle 114 may be configure to provide steering control of the ICE catheter 104. In some embodiments, the handle 114 can provide certain signals to control the ICE catheter 104. The control system 116 can include non-transitory memory configured with instructions, and one or more processors that are configured to execute the instructions to perform operations related to determining the presence of a comet tail aberration and minimizing the appearance of a comet tail aberration in ICE images. Some examples, the control system 116 can include components illustrated in the computer system of FIG. 8.

In operation, the control system 116 provides control signals to the ICE catheter 104 to control generating ICE images and receives ICE image information from the ICE catheter 104. The control system 116 processes the ICE image information, generates displayable ICE images, and provide the ICE images to the display 120. As explained further below, the control system 116 can be configured to determine from the image information the presence of a comet tail aberration, or other image aberrations. The control system 116 can also be configured to, in response to determining the presence of a comet tail aberration, provide control signals to the ICE catheter 104 for minimizing the appearance of it, until aberration in a generated ICE image. The control system 116 may also be configured to, in response to determining the presence of the, until aberration, performing image processing operation to minimize the appearance of the, until aberration in the image.

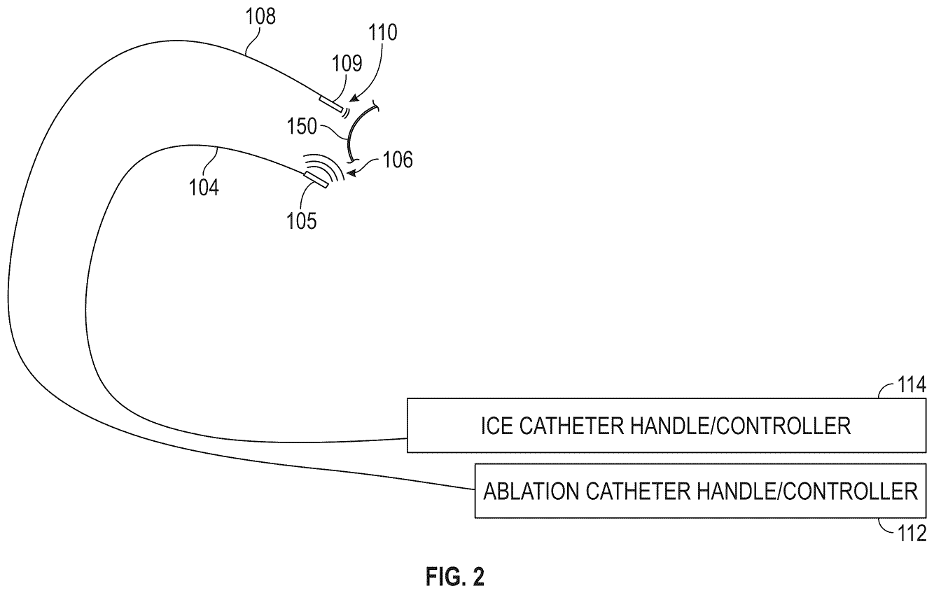

FIG. 2 is a schematic that illustrates an example of a portion of an ICE imaging system that is used to image an RF ablation catheter tip and tissue being ablated. In FIG. 2, the ICE catheter handle/controller 114 is connected to an ICE imaging catheter 104, which includes at its distal tip an ICE imaging element 105. The ablation catheter handle/controller 112 is connected to an ablation catheter 108, which includes at its distal tip an ablation element 109. In operation, the ablation element 109 is positioned near a tissue 150 that is desired to be ablated using energy 110. The ICE imaging element 105 is positioned near the ablation element 109 such that a field of view of the ICE imaging element 105 can include the ablation element 109 and at least a portion of the tissue 150 where the ablation operation will occur. The ICE imaging element 105 produces ultrasound energy 106, transmitting the energy 116 in a series of pulses at a certain scan frequency, and then receives the reflected signals that are the result of the energy 116 reflecting from the ablation catheter tip or ablation element 109 and tissue in the field of view. The received signals are communicated to the control system 116, which generates ICE images and communicates the ICE images to the display 120 for viewing by a medical practitioner.

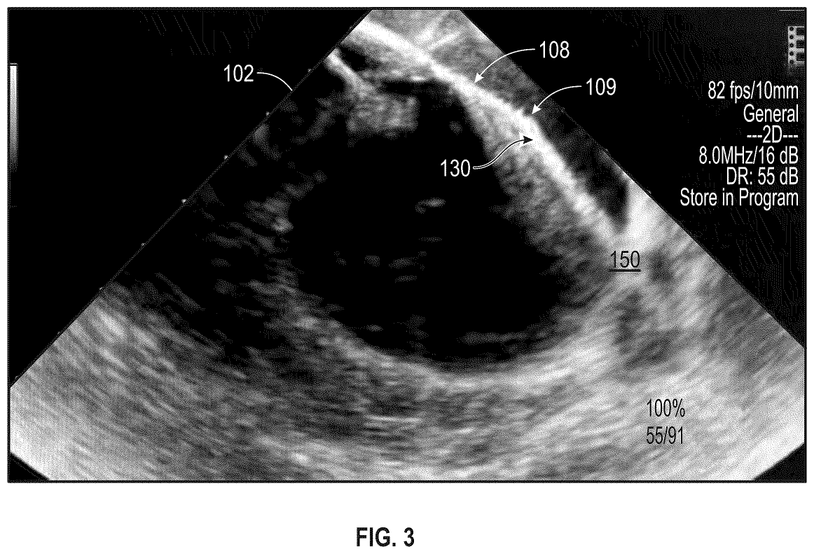

FIG. 3 is an example of an ICE image 102 produced by an ICE imaging system illustrating an RF ablation catheter 108 and an example of a comet tail aberration (also referred to herein as “comet tail” or “reverberation noise”) 130. As illustrated in this image, the comet tail aberration 130 extends from the ablation catheter tip 109 and is positioned between the ablation catheter tip 109 and tissue 150. The comet tail aberration 130 can obscure the position of the ablation catheter tip 109 such that determining the precise location of the ablation catheter tip 109 is difficult, or even impossible.

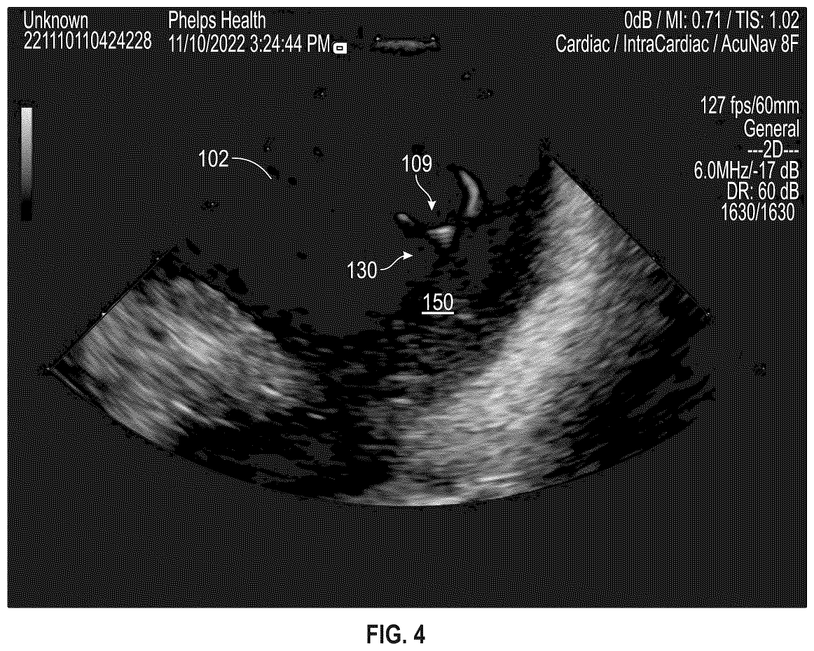

FIG. 4 is an example of an ICE image 102 generated at a 6 MHz scan frequency, which is well matched to the transducer center frequency. FIG. 4 also illustrates reverberation noise (i.e., a comet tail) 130 between the location of the ablation catheter tip 109 and tissue 150 near the ablation catheter tip 109. In FIG. 4 the reverberation noise 130 manifests as a distinct set of lines extending from the ablation catheter tip 109, but it of a relatively short length (for example, compared to the comet tail aberration 109 illustrated in FIG. 3).

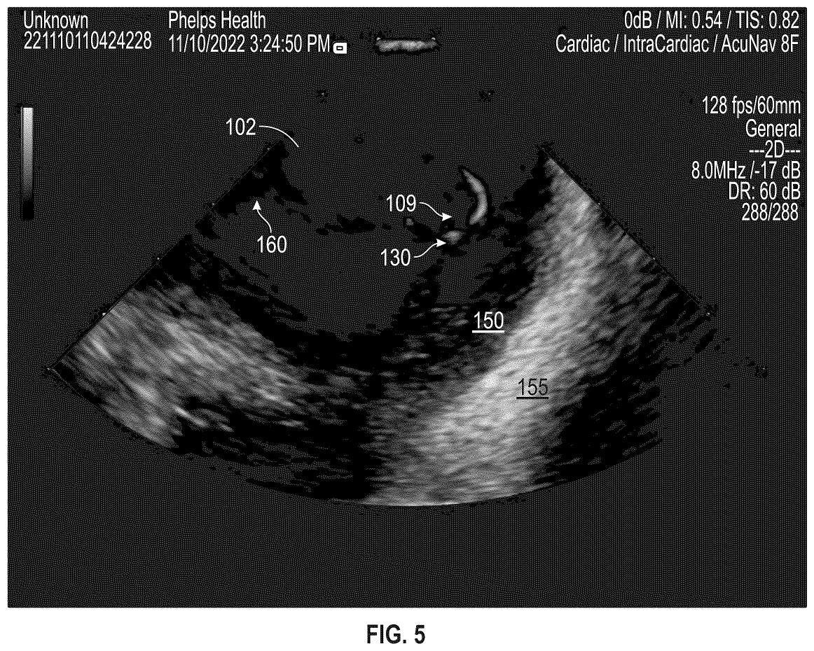

FIG. 5 is an example of an ICE image 102 generated at 8 MHz frequency scan setting, which is close to the upper band edge of the ICE transducer response. Compared to the ICE image shown in FIG. 4, the ICE image shown in FIG. 5 includes a less visible reverberation noise (comet tail aberration) 130. The less visible aberration may be due to the higher frequency of 8 MHz being farther away from the resonant frequency of the ablation catheter. However, this reduced frequency is not desirable because there can be less penetration, loss of axial detail (e.g., coarser tissue texture as seen in area 155), and possible emergence of grating lobe noise 160.

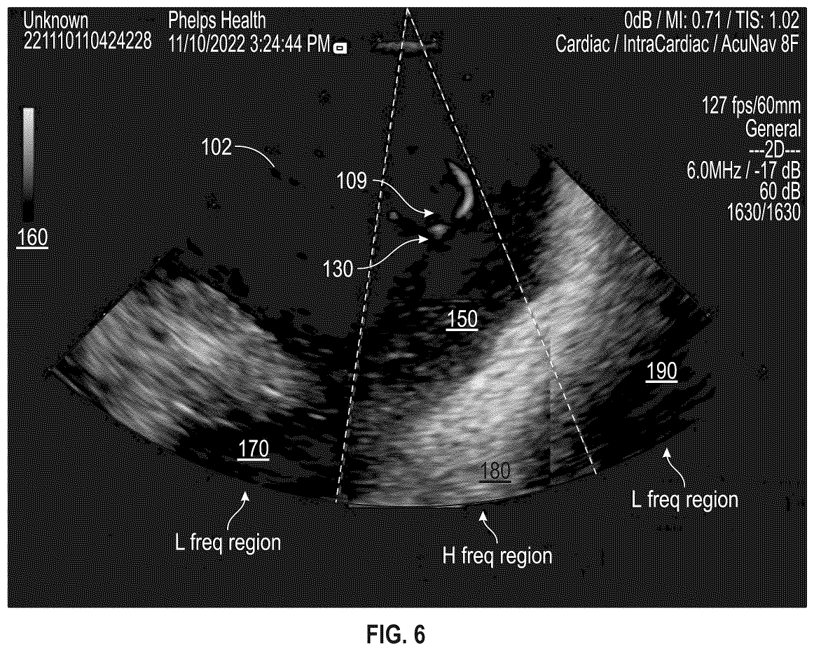

FIG. 6 is another example of an ICE image 102 illustrating a comet tail 130 between the location of the catheter tip 109 and tissue 150 near the catheter tip, where the ICE image 102 is generated using multiple frequencies. In this embodiment, the ICE image 102 includes three imaging zones: a first zone 170, a second zone 180, and a third zone 190. In this example, the first zone 170 is a low frequency imaging zone, that is, it was generated with a low scan frequency imaging characteristic; the second zone 180 is a high frequency imaging zone, that it, it was generated with a high scan frequency imaging characteristic; and the third zone 190 is a second low frequency imaging zone, that is, it was generated with a low scan frequency imaging characteristic. The use of “low” or “high” are relative terms, such that the low scan frequency is lower than the high scan frequency. For example, in certain embodiments of an image generated with multiple scan frequencies, the low scan frequency can be about 6 MHz. in some embodiments, the low scan frequency can be less than 6 MHz. In some embodiments, the high scan frequency can be at or about 7 MHz, or at or about 8 Mhz. In some embodiments, the high scan frequency can be greater than about 7 Mhz. In some embodiments, the high scan frequency can be higher than about 8 Mhz. In some embodiments, the low scan frequency can be a frequency that is lower than the high scan frequency.

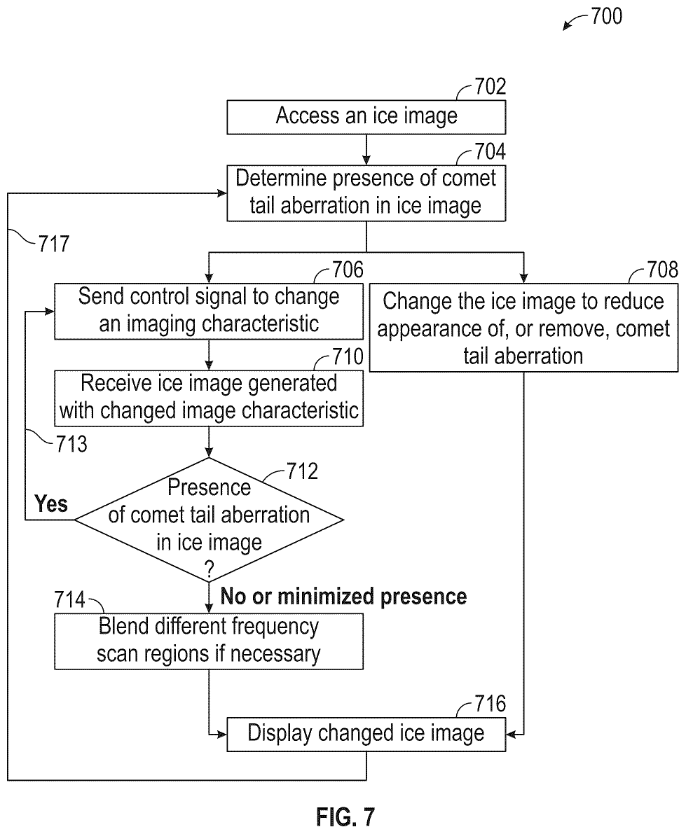

As indicated above, control system 116 (FIG. 1) can be configured to determine the presence of a comet tail aberration, and then the control system 116 can change an imaging characteristic of the ICE catheter and/or perform image processing on the ICE image to reduce or remove the aberration, as described further in connection with the flowchart illustrated in FIG. 7. The control system 116 can be configured with image processing software to determine the presence of a comet tail automatically in ICE images received from the ICE catheter. In an example, the image processing software can use feature detection to determine the location of the ablation catheter tip in an ICE image, and the determine the presence of a comet tail in the ICE image in proximity to the ablation catheter tip. In another example, the image processing software can use feature detection to determine the location of the catheter tip in an ICE image, determine the longitudinal axis of the catheter/catheter tip, and determine the presence of a comet tail in the ICE image in proximity to the catheter tip along the longitudinal axis. In another example, ICE images received from the ICE catheter are displayed on the control panel 118, and a user input indicating the (general) location of the comet tail is received via a user interface on the control panel. For example, by touching a point on the display indicating a location of the comet tail on the displayed image, or by drawing a circle or box around the location of the comet tail on the control panel.

FIG. 7 is a flowchart of a process 700 for minimizing or reducing a comet tail aberration in an ICE image. The process 700 can be performed by the control system 116, which may receive user inputs from the control panel 118. At block 702, the process 700 accesses an ICE image, or a series of ICE images. In some embodiments, the ICE image can be a standard B-mode image for a given ICE procedure. The ICE image may be received from an ICE catheter coupled to the control system 116. In some embodiments, the ICE image is accessed from a stored location. For example, memory in the control system 116 or memory in another component. At block 704, the process 700 determines the presence of a comet tail aberration in an ICE image. In some embodiments, the control system 116 can be configured to have pattern recognition software running in the background, the pattern recognition software is configured to determine the location of the ablation catheter tip and determine a comet tail aberration within a certain proximity to the ablation catheter tip 109 (FIG. 1). For example, with a region of interest (ROI) at the distal end of the ablation catheter tip 109. The ROI may surround the ablation catheter tip 109. In another example, the ROI may be located off the end of the ablation catheter tip 109 such that at least a portion of the ROI is between the ablation catheter tip and tissue that is desired to be ablated. The pattern recognition software can configure the control system 116 to automatically detect/flag the presence of an ablation catheter tip 109 and associated comet tail 130 in the ROI. For example, determine from the pixels in the ROI if there is a series of lines that indicate reverberation of the ICE energy between the ablation catheter tip and the tissue, or if there is an area of pixels extending from the ablation catheter tip indicative of a comet tail. In some embodiments, a machine learning algorithm can be trained using a plurality of ICE images that include comet tails and a plurality of ICE images that do not include comet tails. The portion of an ICE image in the ROI can be used as an input to the trained machine learning algorithm, and the machine learning (ML) algorithm can determine if a comet tail aberration exists based on its similarity to the images the ML algorithm was trained on. In some embodiments, determining the presence of a comet tail aberration can be based at least in part on the use of a priori knowledge of ablation catheter characteristics. In some embodiments, the control system 116 can include reverberation wavefront modeling and pattern recognition algorithms to help determine if a comet tail aberration exists. In some embodiments, determination of the presence of a comet tail can include comparing different images having two different frequencies to determine if the image changes in the location near the catheter tip when different scan frequencies are used, where a change in the image in the ROI can indicate that a comet tail exists in the image. In some embodiments, filtering of the ICE image can be used to determine if a comet tail aberration exists. For example, a morphological filter and/or image segmentation methods related to the shape of the comet tail aberration can be used to determine if a comet tail aberration exists.

In response to determining that a comet tail aberration exists in the ICE image, the process 700 may proceed to block 708 where image processing may reduce or remove comet tail aberration such that it is less visible or not visible in displayed images. Or process 700 may proceed to block 706 where the control system 116 provides a control signal to the catheter 104 to change an imaging characteristic of the ICE catheter. For example, the control system 116 may provide a signal to the ICE catheter to switch to a different imaging characteristic. For example, switch to a different B-mode acquisition scan sequence (e.g., scan parameter) and/or image formation processing for a ROI that covers the catheter and tissue ablation region. The ROI may define an entire B-image sector, or a box around the catheter tip like a color Doppler (CD) ROI. Feedback loops may be employed to adjust scan parameters based on image analysis results and pre-defined criteria for comet tail suppression. If no feedback loop is needed, the overall frames per second (FPS) would stay the same as for the preset. The changed scan parameters may include one or more of transmission (Tx) focus location, Tx frequency (fundamental or harmonic), Tx pulse sequence (single pulse or coded excitation), and/or use of a different beamforming method tuned towards reverb noise isolation. A decision for the process 700 to proceed to block 706 or 708 be based on a user input or based on an automatic determination of how bad the aberration is. For example, a decision to proceed to block 706 or 708 may depend on a level of how visible the comet tail aberration is in the ICE image. For example, if the ICE image is not very strong (e.g., below a user perceived level or below a threshold), the process 700 may proceed to block 708 where filtering or other image processing may reduce the appearance of the comet tail in the image, where if the comet tail aberration in the ICE image is strong such that it obscures the location of the catheter tip (either above a user's perceived level of hinderance or above a threshold value) the process 700 may proceed to block 706.

After the process 700 proceeds to block 706, the process proceeds to block 710 where one or more ICE images are received, and the received images being generated with the changed imaging characteristic. At block 712, the process 700 again determines whether or not there is a comet tail present in the ICE image. If at block 712 the control system determines there is no longer a comet tail aberration in the ICE image or the aberration has been sufficiently reduced, the process proceeds to block 714. Blocks 706, 710, and 712 can be executed as a loop, where at block 712 in response to the control system 116 determining the presence of a comet tail in the image (e.g., any comet tail or a comet tail of a certain amount), the process 700 proceeds on loop 713 back to block 706.

In the loop of blacks 706, 710, and 712, the control system 116 is configured to determine a change to an imaging characteristic, or more than one imaging characteristics, of the ICE catheter and provide a control signal to the ICE catheter at block 706, receive images generated by the ICE catheter using the changed imaging characteristic(s) at block 710, and evaluate the received images to determine if the image generated with the changed imaging characteristic(s) produced an image with a comet tail at block 712. In some embodiments, the control system 116 can be configured to execute this loop rapidly, rapidly evaluating the stream of images produced by the ICE catheter without user intervention. During this loop, the ICE images can be displayed, and in some embodiments a user input is received to indicate whether or not the current or changed imaging characteristics used to produce the ICE images are sufficient to remove or minimize the comet tail aberration. Referring to block 706, the imaging characteristic can be changed based at least in part on image analysis of the ICE image, information (e.g., operational specifications) relating to the ICE catheter being used, and/or predefined criteria relating to comet tail suppression. In some embodiments, a machine learning process is used to optimize the process of changing the imaging characteristic. The imaging characteristic can include a scan parameter. For example, one or more of transmission focus, transmission frequency (in fundamental or harmonic imaging mode), transmission pulse sequence (e.g., single pulse of coded excitation), and/or use of a different beamforming method which is developed or tuned to minimize reverberation noise/aberrations.

Referring to block 712, the control system 116 can be configured to determine if the image generated with the changed imaging characteristic(s) produced a better image having a less visible comet tail (e.g., compared to one or more previous images). In some embodiments, the control system 116 is configured to determine if the image generated with the changed imaging characteristic(s) produces an image with a less visible comet tail aberration without detrimentally affecting other portions of the image. After image characteristics have been changed to sufficiently remove or reduce the comet tail aberration as determined in block 712, at block 714 the control system can blend boundaries of regions that were scanned with different frequencies. For example, the first low frequency scan region 170, the high frequency scan frequency region 180, and the second low frequency scan region 190 illustrated in FIG. 6. In some embodiments, the blending functionality of block 714 can be performed in the loop of blocks 706, 710, and 712.

In the illustrated embodiment, whether the process 700 used image processing techniques to reduce or remove the comet tail aberration at block 708, or if the process 700 changed one or more imaging characteristics to reduce or remove the comet tail aberration in blocks 706, 710, 712, and 714, the process 700 continues to block 716 where the resulting ICE images are displayed. As shown in FIG. 7, process 700 can continue during the procedure to improve the quality of the ICE image displayed to the medical practitioner by proceeding to loop back to block 704 where again the received ICE images are evaluated. In this way, the process 700 can provide continuous evaluating and improving of the received and displayed ICE images even when conditions are changing during a procedure.

FIG. 8 is a block diagram that illustrates a computer system 800 with which certain methods discussed herein may be implemented. Computer system 800 includes a bus 802 or other communication mechanism for communicating information, and a hardware processor, or multiple processors, 804 coupled with bus 802 for processing information. Hardware processor(s) 804 may be, for example, one or more general purpose microprocessors.

Computer system 800 also includes a main memory 806, such as a random-access memory (RAM), cache and/or other dynamic storage devices, coupled to bus 802 for storing information and instructions to be executed by processor 804. Main memory 806 also may be used for storing temporary variables or other intermediate information during execution of instructions to be executed by processor 804. Such instructions, when stored in storage media accessible to processor 804, render computer system 800 into a special-purpose machine that is customized to perform the operations specified in the instructions. The main memory 806 may for example, include instructions to implement a user interface as illustrated in FIG. 6, calculate data metrics, allow a user to filter data and change data in data sets, and store information indicting the operations performed to clean and/or prepare data to a log the data being stored in some examples in data objects as defined by an ontology.

Computer system 800 further includes a read only memory (ROM) 808 or other static storage device coupled to bus 802 for storing static information and instructions for processor 804. A storage device 810, such as a magnetic disk, optical disk, or USB thumb drive (Flash drive), etc., is provided and coupled to bus 802 for storing information and instructions.

Computer system 800 may be coupled via bus 802 to a display 812, such as a cathode ray tube (CRT) or LCD display (or touch screen), for displaying information to a computer user. An input device 814, including alphanumeric and other keys, is coupled to bus 802 for communicating information and command selections to processor 804. Another type of user input device is cursor control 816, such as a mouse, a trackball, or cursor direction keys for communicating direction information and command selections to processor 804 and for controlling cursor movement on display 812. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

Computing system 800 may include a user interface module to implement a GUI that may be stored in a mass storage device as computer executable program instructions that are executed by the computing device(s). Computer system 800 may further, as described below, implement the techniques described herein using customized hard-wired logic, one or more ASICs or FPGAs, firmware and/or program logic which in combination with the computer system causes or programs computer system 800 to be a special-purpose machine. According to one embodiment, the techniques herein are performed by computer system 800 in response to processor(s) 804 executing one or more sequences of one or more computer readable program instructions contained in main memory 806. Such instructions may be read into main memory 806 from another storage medium, such as storage device 810. Execution of the sequences of instructions contained in main memory 806 causes processor(s) 804 to perform the process steps described herein. In alternative embodiments, hard-wired circuitry may be used in place of or in combination with software instructions.

Various forms of computer readable storage media may be involved in carrying one or more sequences of one or more computer readable program instructions to processor 804 for execution. For example, the instructions may initially be carried on a magnetic disk or solid-state drive of a remote computer. The remote computer can load the instructions into its dynamic memory and send the instructions over a telephone line using a modem. A modem local to computer system 800 can receive the data on the telephone line and use an infra-red transmitter to convert the data to an infra-red signal. An infra-red detector can receive the data carried in the infra-red signal and appropriate circuitry can place the data on bus 802. Bus 802 carries the data to main memory 806, from which processor 804 retrieves and executes the instructions. The instructions received by main memory 806 may optionally be stored on storage device 810 either before or after execution by processor 804.

Computer system 800 also includes a communication interface 818 coupled to bus 802. Communication interface 818 provides a two-way data communication coupling to a network link 820 that is connected to a local network 822. For example, communication interface 818 may be an integrated services digital network (ISDN) card, cable modem, satellite modem, or a modem to provide a data communication connection to a corresponding type of telephone line. As another example, communication interface 818 may be a local area network (LAN) card to provide a data communication connection to a compatible LAN (or WAN component to communicate with a WAN). Wireless links may also be implemented. In any such implementation, communication interface 818 sends and receives electrical, electromagnetic or optical signals that carry digital data streams representing various types of information.

Network link 820 typically provides data communication through one or more networks to other data devices. For example, network link 820 may provide a connection through local network 822 to a host computer 824 or to data equipment operated by an Internet Service Provider (ISP) 826. ISP 826 in turn provides data communication services through the worldwide packet data communication network now commonly referred to as the “Internet” 828. Local network 822 and Internet 828 both use electrical, electromagnetic or optical signals that carry digital data streams. The signals through the various networks and the signals on network link 820 and through communication interface 818, which carry the digital data to and from computer system 800, are example forms of transmission media.

Computer system 800 can send messages and receive data, including program code, through the network(s), network link 820 and communication interface 818. In the Internet example, a server 830 might transmit a requested code for an application program through Internet 828, ISP 826, local network 822 and communication interface 818.

The received code may be executed by processor 804 as it is received, and/or stored in storage device 810, or other non-volatile storage for later execution.

Accordingly, as an example, in some embodiments, of the computer system 800, the computer system comprises a first non-transitory computer storage medium storage device 810 configured to under control of a computing system comprising one or more computer processors configured to execute specific instructions, to perform a method comprising receiving at the computer system an intravascular ultrasound (ICE) imaging system on a catheter coupled to the computer system, ICE images depicting the ablation catheter tip and an area proximate to the ablation catheter tip, automatically determining, by the computer system, a location of the tip of the ablation catheter in the received ICE images, automatically determining, by the computer system, the presence of a comet tail aberration in a region of interest (ROI) near the ablation catheter tip in the received ICE images, and in response to determining the aberration is present in the ROI, automatically changing, by the computer system, an imaging characteristic of the ICE imaging system to affect the appearance of the aberration in the ROI in subsequent images generated by the ICE imaging system using the changed imaging characteristic.

### Implementation on a Computer System

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure. For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums).

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression, or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide user interface functionality, such as a graphical user interface (“GUI”), among other things.

### EXAMPLES OF CERTAIN EMBODIMENTS

Embodiment 1. A computer system implemented method of reducing a comet tail aberration in an image of an ablation catheter, the method comprising: receiving, by the computer system from an intracardiac echocardiography (ICE) imaging system on a catheter coupled to the computer system, a first set of images depicting the ablation catheter tip and an area proximate to the ablation catheter tip, the first set of images including at least one ICE image; automatically determining, by the computer system, a location of the tip of the ablation catheter in the first set of images; automatically determining, by the computer system, the presence of a comet tail aberration in a region of interest (ROI) near the ablation catheter tip in the first set of images; and in response to determining the aberration is present in the ROI, automatically changing, by the computer system, an imaging characteristic of the ICE imaging system to affect the appearance of the aberration in the ROI in subsequent images generated by the ICE imaging system using the changed imaging characteristic.

Embodiment 2. The method of embodiment 1, wherein changing the imaging characteristic comprises changing a scan parameter used to generate the first set of images.

Embodiment 3. The method of embodiment 2, wherein changing the scan parameter comprises changing transmission focus location of the ICE imaging system.

Embodiment 4. The method of embodiment 2, wherein changing the scan parameter comprises changing a transmission frequency of the ICE imaging system.

Embodiment 5. The method of embodiment 2, wherein changing the scan parameter comprises changing a fundamental transmission frequency of the ICE imaging system.

Embodiment 6. The method of embodiment 2, wherein changing the scan parameter comprises changing a harmonic mode transmission frequency of the ICE imaging system.

Embodiment 7. The method of embodiment 2, wherein changing a scan parameter comprises changing a transmission pulse of the ICE imaging system.

Embodiment 8. The method of embodiment 7, wherein changing the transmission pulse comprises a single pulse sequence transmitted by the ICE imaging system.

Embodiment 9. The method of embodiment 7, wherein changing the transmission pulse comprises coded excitation pulses transmitted by the ICE imaging system.

Embodiment 10. The method of embodiment 1, wherein changing the imaging characteristic comprises changing a beamforming method of the ICE imaging system.

Embodiment 11. The method of embodiment 1, wherein changing the imaging characteristic comprises changing a beamforming method tuned towards reverberation noise isolation.

Embodiment 12. The method of embodiment 1, wherein changing the imaging characteristic comprises changing a beamforming method to a model-based approach that is tuned towards reverberation noise or wavefront isolation.

Embodiment 12. The method of embodiment 1, wherein the aberration is a noise artifact along the ultrasound scan line that extends beyond the tip of the ablation catheter.

Embodiment 13. The method of embodiment 1, further comprising: receiving a second set of images from the ICE imaging system that were generated using the changed imaging characteristic, the second set of images including at least one ICE image; automatically determining in the ROI, in images in the second set of images, the presence of the comet tail aberration; determining if the aberration has been reduced; in response to determining that the aberration has not been reduced, automatically changing a second imaging characteristic of the ICE imaging system to change the appearance of the comet tail aberration in the ROI in subsequent images generated by the ICE system using the second imaging characteristic.

Embodiment 14. The method of embodiment 1, further comprising generating and displaying a representation of the ICE images.

Embodiment 15. The method of embodiment 1, in response to a user input, changing a second imaging characteristic of the ICE imaging system to change the appearance of the comet tail aberration in the ROI in subsequent images generated by the ICE system using the second imaging characteristic.

Embodiment 16. The method of embodiment 1, wherein automatically determining, by the computer system, the presence of the comet tail aberration in the ROI comprises accessing predetermined information of the ablation catheter characteristics.

Embodiment 17. The method of embodiment 1, wherein automatically determining, by the computer system, the presence of the comet tail aberration in the ROI comprises using reverb wavefront modeling.

Embodiment 18. The method of embodiment 1, wherein automatically determining, by the computer system, the presence of the comet tail aberration in the ROI comprises comparing ICE images generated using two different frequencies.

Embodiment 19. The method of embodiment 1, wherein automatically determining, by the computer system, the presence of the comet tail aberration in the ROI comprises comparing ICE images generated using two different imaging modes.

Embodiment 20. The method of embodiment 1, wherein automatically determining, by the computer system, the presence of the comet tail aberration in the ROI comprises using one or more morphological filters or other image segmentation methods.

Embodiment 21. The method of embodiment 1, wherein automatically determining, by the computer system, the presence of the comet tail aberration in the ROI comprises using a machine learning (ML) process.

Embodiment 22. A non-transient computer readable medium containing program instructions for causing a computer to perform the method of reducing a comet tail aberration in an image of an ablation catheter, the method comprising: under control of a computing system comprising one or more computer processors configured to execute specific instructions, receiving at the computer system an intracardiac echo (ICE) imaging system on a catheter coupled to the computer system, a first set of images depicting the ablation catheter tip and an area proximate to the ablation catheter tip, the first set of images including at least one ICE image; automatically determining, by the computer system, a location of the tip of the ablation catheter in the first set of images; automatically determining, by the computer system, the presence of a comet tail aberration in a region of interest (ROI) near the ablation catheter tip in the received ICE images; and in response to determining the aberration is present in the ROI, automatically changing, by the computer system, an imaging characteristic of the ICE imaging system to affect the appearance of the aberration in the ROI in subsequent images generated by the ICE imaging system using the changed imaging characteristic.

Embodiment 23. A computer system implemented method of reducing a comet tail aberration in an image of an ablation catheter, the method comprising: receive and display a first ICE image, wherein the first ICE image is generated with first imaging characteristics;

determine the location of a catheter tip in the first ICE image; determine the presence of a comet tail aberration, associated with the catheter tip, in a region of interest (ROI) in the ICE image; in response to determining the presence of the comet tail aberration, initiate a dual frequency B-mode scanning sequence where a first frequency is used to generate a second ICE image in a first region of interest (ROI) that covers the catheter tip and a second region of interest (ROI) that includes a tissue ablation region; process the second ICE image to generate a smoothed ICE image, the smoothed ICE image having smoothed boundaries between a background region, the first ROI, and the second ROI; and display the smoothed second ICE image.

Embodiment 24. A computer system implemented method of reducing a comet tail aberration in an image of an ablation catheter, the method comprising: receive and display a first ICE image, wherein the first ICE image is generated with first imaging characteristics; determine the location of a catheter tip in the first ICE image; determine the presence of a comet tail aberration, associated with the catheter tip, in a region of interest (ROI) in the ICE image; in response to determining the presence of the comet tail aberration, initiate a dual frequency B-mode scanning sequence where a first frequency is used to generate a normal background ICE image with the desired image quality in terms of spatial resolution and penetration, and a second frequency is used for a special region of interest (ROI) that covers the catheter tip and a tissue ablation region; the second frequency is chosen specifically to minimize the comet tail artifact even if the general image quality may be compromised (within an acceptable level); blend the image regions generated by the two frequencies together by smoothing the boundaries between the background region and the special ROI; and display the blended ICE image.

The foregoing embodiments are examples of certain embodiments of this disclosure, however, these are not limiting and other embodiments are also possible. Many variations and modifications may be made to the embodiments described herein, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of terminology when describing certain features or aspects of the systems and methods should not be taken to imply that the terminology is being re-defined herein to be restricted to including any specific characteristics of the features or aspects of the systems and methods with which that terminology is associated.

### TERMINOLOGY

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps but may include additional steps. When used in the context of a device, the term “comprising” means that the device includes at least the recited features or components but may also include additional features or components. Also, the term “or” is used in its inclusive sense (and not in its exclusive sense) so that when used, for example, to connect a list of elements, the term “or” means one, some, or all the elements in the list. Further, the term “each,” as used herein, in addition to having its ordinary meaning, can mean any subset of a set of elements to which the term “each” is applied.

Conditional language, such as “can,” “could,” “might,” or “may” unless specifically stated otherwise, or otherwise understood within the context as used, is intended to convey that certain embodiments include, while other embodiments do not include, certain features, elements, or steps. Thus, such conditional language is not intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning, which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a user; however, they can also include any third-party instruction of those actions, either expressly or by implication.

Conditional language used herein, such as, among others, “can,” “could,” “might,” “may” “e.g.,” and the like, unless specifically stated otherwise, or otherwise understood within the context as used, is intended to convey that certain, certain features, elements and/or steps are optional. Thus, such conditional language is not intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a user; however, they can also include any third-party instruction of those actions, either expressly or by implication.

The methods and tasks described herein may be performed and fully automated by a computer system. The computer system may in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry of the computer system. Where the computer system includes multiple computing devices, these devices may but need not, be co-located. The results of the disclosed methods and tasks may be persistently stored by transforming physical storage devices, such as solid-state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A computer system implemented method of reducing a comet tail aberration in an image of an ablation catheter, the method comprising:
receiving, by the computer system from an intracardiac echocardiography (ICE) imaging system on a catheter coupled to the computer system, a first set of images depicting an ablation catheter tip and an area proximate to the ablation catheter tip, the first set of images including at least one ICE image;
automatically determining, by the computer system, a location of the tip of the ablation catheter in the first set of images;
automatically determining, by the computer system, a presence of a comet tail aberration in a region of interest (ROI) near the ablation catheter tip in the first set of images; and
in response to determining the aberration is present in the ROI, automatically changing, by the computer system, an imaging characteristic of the ICE imaging system to affect an appearance of the aberration in the ROI in subsequent images generated by the ICE imaging system using the changed imaging characteristic.

2. The method of claim 1, wherein changing the imaging characteristic comprises changing a scan parameter used to generate the first set of images.

3. The method of claim 2, wherein changing the scan parameter comprises changing transmission focus location of the ICE imaging system.

4. The method of claim 2, wherein changing the scan parameter comprises changing a transmission frequency of the ICE imaging system.

5. The method of claim 2, wherein changing a scan parameter comprises changing a transmission pulse of the ICE imaging system.

6. The method of claim 2, wherein changing a scan parameter comprises changing a transmission pulse of the ICE imaging system.

7. The method of claim 1, wherein changing the imaging characteristic comprises changing a beamforming method of the ICE imaging system.

8. The method of claim 1, wherein changing the imaging characteristic comprises changing a beamforming method tuned towards reverberation noise isolation.

9. The method of claim 1, wherein changing the imaging characteristic comprises changing a beamforming method to a model-based approach that is tuned towards reverberation noise or wavefront isolation.

10. The method of claim 1, wherein the aberration is a noise artifact along an ultrasound scan line that extends beyond the tip of the ablation catheter.

11. The method of claim 1, further comprising:
receiving a second set of images from the ICE imaging system that were generated using the changed imaging characteristic, the second set of images including at least one ICE image;
automatically determining in the ROI, in images in the second set of images, the presence of the comet tail aberration;
determining if the aberration has been reduced; and
in response to determining that the aberration has not been reduced, automatically changing a second imaging characteristic of the ICE imaging system to change the appearance of the comet tail aberration in the ROI in subsequent images generated by the ICE system using the second imaging characteristic.

12. The method of claim 1, in response to a user input, changing a second imaging characteristic of the ICE imaging system to change the appearance of the comet tail aberration in the ROI in subsequent images generated by the ICE system using the second imaging characteristic.

13. The method of claim 1, wherein automatically determining, by the computer system, the presence of the comet tail aberration in the ROI comprises accessing predetermined information of the ablation catheter characteristics.

14. The method of claim 1, wherein automatically determining, by the computer system, the presence of the comet tail aberration in the ROI comprises using reverb wavefront modeling.

15. The method of claim 1, wherein automatically determining, by the computer system, the presence of the comet tail aberration in the ROI comprises comparing ICE images generated using two different frequencies.

16. The method of claim 1, wherein automatically determining, by the computer system, the presence of the comet tail aberration in the ROI comprises comparing ICE images generated using two different imaging modes.

17. The method of claim 1, wherein automatically determining, by the computer system, the presence of the comet tail aberration in the ROI comprises using one or more morphological filters or other image segmentation methods.

18. The method of claim 1, wherein automatically determining, by the computer system, the presence of the comet tail aberration in the ROI comprises using a machine learning (ML) process.

19. A non-transient computer readable medium containing program instructions for causing a computer to perform a method of reducing a comet tail aberration in an image of an ablation catheter, the method comprising:
under control of a computing system comprising one or more computer processors configured to execute specific instructions,
receiving at the computer system an intracardiac echo (ICE) imaging system on a catheter coupled to the computer system, a first set of images depicting an ablation catheter tip and an area proximate to the ablation catheter tip, the first set of images including at least one ICE image;
automatically determining, by the computer system, a location of the tip of the ablation catheter in the first set of images;
automatically determining, by the computer system, a presence of a comet tail aberration in a region of interest (ROI) near the ablation catheter tip in the received ICE images; and
in response to determining the aberration is present in the ROI, automatically changing, by the computer system, an imaging characteristic of the ICE imaging system to affect an appearance of the aberration in the ROI in subsequent images generated by the ICE imaging system using the changed imaging characteristic.

20. A computer system implemented method of reducing a comet tail aberration in an image of an ablation catheter, the method comprising:
receive and display a first ICE image, wherein the first ICE image is generated with first imaging characteristics;
determine a location of a catheter tip in the first ICE image;
determine a presence of a comet tail aberration, associated with the catheter tip, in a region of interest (ROI) in the ICE image;
in response to determining the presence of the comet tail aberration, initiate a dual frequency B-mode scanning sequence where a first frequency is used to generate a second ICE image in a first region of interest (ROI) that covers the catheter tip and a second region of interest (ROI) that includes a tissue ablation region;
process the second ICE image to generate a smoothed ICE image, the smoothed ICE image having smoothed boundaries between a background region, the first ROI, and the second ROI; and
display the smoothed second ICE image.
